(19) United States
(12) Patent Application Publication
(10) Pub. No.: US 2005/0255509 A1
(43) Pub. Date: Nov. 17, 2005

(54) In vivo measurement of the relative fluxes through ribonucleotide reductase vs. deoxyribonucleoside pathways using isotopes

(75) Inventors: Marc K. Hellerstein, Kensington, CA (US); Scott M. Turner, Oakland, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: KineMed, Inc., Emeryville, CA (US)

(21) Appl. No.: 11/095,029

(22) Filed: Mar. 30, 2005

Related U.S. Application Data
(60) Provisional application No. 60/558,215, filed on Mar. 30, 2004.

(51) Int. Cl. C12Q 1/68

(52) U.S. Cl. 435/6

(57) ABSTRACT

The methods of the present invention allow for the measurement of ribonucleotide reductase (RR) activity, an important enzyme in the de novo DNA synthesis pathway. Ribonucleotide reductase converts all four ribonucleotides to their deoxy form and is a rate-controlling step in this pathway. Biosynthetic pathways of deoxyribonucleotides (dN) have received considerable attention in the context of anti-proliferative chemotherapy. Inhibitors of various steps in dN biosynthesis, including inhibitors of RR are among the most useful chemotherapeutic agents in cancer, viral infections, and other therapeutic uses. DNA synthesis from the dN salvage pathway is also an important component to DNA replication. The relative contributions from RR vs. salvage pathways are critical to the actions and effectiveness of chemotherapeutic agents that act on nucleoside metabolic pathways. Until now, however, it has not been possible to study these metabolic processes in vivo. Disclosed within are methods of measuring RR activity in vivo and in vitro which find use, among other things, in drug discovery, development, and approval.

### CROSS REFERENCE TO RELATED APPLICATIONS

This application claims priority to U.S. provisional application No. 60/558,215 filed on Mar. 30, 2004, which is herein incorporated by reference in its entirety.

### FIELD OF THE INVENTION

The present invention relates to methods for measuring cell proliferation and changes in cell proliferation. More specifically, the methods allow assessment of ribonucleotide reductase activity by comparing the incorporation of labeled purine deoxynucleosides such as deoxyadenosine with labeled pyrimidine deoxynucleosides such as deoxythymidine into nucleic acids, particularly DNA.

### BACKGROUND OF THE INVENTION

Nucleoside metabolism is central to a number of aspects of cellular function, including ribonucleic acid (RNA) synthesis, deoxyribonucleic acid (DNA) replication, and metabolism of high-energy phosphates such as adenosine triphosphate (ATP). Biosynthetic pathways of deoxyribonucleotides (dN), in particular, have received considerable attention in the context of anti-proliferative chemotherapy. Inhibitors of various steps in dN biosynthesis, including inhibitors of ribonucleotide reductase (RR) and dN salvage (e.g., thymidine kinase, deoxycytidine kinase) are among the most useful chemotherapeutic agents in cancer, viral infections, and other therapeutic uses.

Ribonucleotide reductase is a rate-controlling step in the de novo nucleotide synthesis pathway (DNNSP, FIG. 1A) and exhibits several very interesting regulatory features. A single RR enzyme catalyzes the conversion of all four ribonucleotide diphosphates (NDPs, FIG. 1A) to dN-diphosphates (dNDPs). This shared control is remarkable, however, in that RR demonstrates different feed-back regulation for purine as compared to pyrimidine NDPs. Increased concentration of pyrimidine dNTPs, (dCTP or dTTP) allosterically inhibits RR activity for the substrate pyrimidine NDPs (CDP, UDP) but stimulates RR activity for purine NDPs (ADP, GDP) (FIG. 1B). The apparent logic of this regulatory network may be to ensure matching availability of all four dNTPs (since all are necessary for DNA replication) when pyrimidine dNTP concentrations change.

A metabolic consequence of this regulatory architecture is that the DNNSP contribution to pyrimidine dNTPs is variable and dependent upon extracellular availability of pyrimidine nucleosides, whereas the DNNSP contribution to purine dNTPs is always dominant and essentially constant. Put differently, the DNNSP input into pyrimidine dNTPs is suppressible and inducible, while the DNNSP contribution to purine dNTPs is constitutive.

The greater capacity to take up exogenous pyrimidine than purine nucleosides is the technical reason why classic exogenous DNA labeling techniques, such as ³H-dT or BrdU, have utilized labeled pyrimidine nucleosides, not purines.

The relative contributions from RR vs. salvage pathways are therefore critical to the actions and effectiveness of chemotherapeutic agents that act on nucleoside metabolic pathways. Until now, however, it has not been possible to study these metabolic processes in vivo. Estimates of RR or dN-salvage activity have largely been based on ex vivo studies of enzyme content or activity. This gap in methodology represents an important limitation for evaluating the mechanism of action or efficacy of agents such as RR inhibitors (e.g., hydroxyurea, gemcitabine, tezacitabine) or dN kinase inhibitors, of particular concern is to distinguish in vivo actions for agents with more than putative activity, such as gemcitabine, which is reported to have both RR inhibitory and DNA chain-terminating activities. Distinguishing between these actions in vivo in a tumor can be critical for evaluating drug resistance of cancers, predicting clinical response, devising combined therapeutic regimens, etc.

Establishing the rate-controlling role of RR or dN kinases in S-phase DNA synthesis (i.e., cell division) would also be very useful for drug development and evaluation. An inhibitor of RR might be active but not impair DNA synthesis if, for example, dN salvage could compensate in a particular cell type. At present, there is no means of identifying this scenario as opposed to simple failure of the RR inhibitor to inhibit flux through RR.

The availability of a screening tool for identifying or confirming agents with activity on RR or dN salvage pathways would also be of potential utility for drug discovery and development.

Disclosed herein are methods that allow for the contribution to DNA replication from RR (through DNNSP, FIG. 1) relative to that from dN salvage (FIG. 1) to be determined in vivo. The rate of DNA replication (i.e., cell division) can be measured concurrently.

### SUMMARY OF THE INVENTION

Accordingly, in one aspect, the present invention provides methods for measuring ribonucleotide reductase activity in a living system. The methods comprise administering an isotope-labeled substrate to the living system for a period of time sufficient for the substrate to be incorporated into at least one type of purine deoxynucleotide and at least one type of pyrimidine deoxynucleotides, to form labeled purine and pyrimidine deoxynucleotides, respectively, within a DNA molecule in the living system. Optionally, several administrations for different time periods can also be done. A first sample is obtained from the living system; again, optionally multiple samples can be obtained. The amount of labeled purine deoxynucleotides and labeled pyrimidine deoxynucleotides derived from said DNA derived from said sample is then quantified. Then the amount of labeled purine and pyrimidine deoxynucleotides is compared to the amount of labeled purine and pyrimidine deoxynucleotides observed in a control living system to determine a difference in ribonucleotide reductase activity in the living system as compared to a control living system. In some aspects, the pyrimidine deoxynucleotide is deoxythymidine and the purine deoxynucleotide is deoxyadenosine.

In another aspect, the invention provides methods as outlined above where the molecular flux rates of the labeled purine and pyrimidine deoxynucleotides are calculated. The ratio of flux rates is then calculated and compared to the molecular flux rate ratio of a control living system.

In a further aspect, the invention provides methods that optionally further comprise administering a candidate agent to the living system, either before, during or after the administration of the isotope-labeled substrate. Optional embodiments utilize a first determination of RR activity in the absence of the candidate agent in a subject and a second determination after the administration of the candidate agent. Optional further embodiments utilize the administration of different concentrations of candidate agents.

In an additional aspect, the invention optionally provides more than one administration of isotope-labeled substrate, e.g. multiple discontinuous administrations. The determination of molecular flux rates, ratios and/or RR activity can be done for each administration.

In a further aspect, the present invention allows for the measurement of ribonucleotide reductase (RR) activity. Ribonucleotide reductase activity is measured by comparing isotopically-labeled deoxyadenosine with isotopically-labeled deoxythymidine in DNA molecules isolated from cells and tissues of subjects given isotopically-labeled substrates for deoxynucleotides.

Compounds or conditions that inhibit RR activity will be detected by changes in ratios of dA/dT in subjects to that of ratios of dA/dT in control subjects. This is so because when RR is inhibited, the activity of thymidine kinase (tk) is increased. This results in increased salvage of thymidine and reduced relative contribution from de novo thymidine synthesis into DNA. Traditionally, this effect is assayed in vitro by measuring the increase in the concentration of the free (thymidine triphosphate (TTP)) pool relative to other deoxynucleotides. In contrast to tk, basal deoxyadenosine kinase (dAk) activity is low and is not up-regulated in response to RR inhibition. The salvage of deoxyadenosine (dA) is very low in normal cells allowing for the measurement of DNA synthesis from the incorporation of de novo deoxyadenosine; furthermore, since dAk activity is not affected by RR inhibition it can be used as the denominator for calculating fractional de novo thymidine synthesis. The relative inhibition of RR can then be inferred from the reduction in de novo thymidine incorporation relative to dA incorporation into DNA.

The isotopic label may be stable or radioactive.

The methods of the present invention find use in all stages of the drug discovery, development, and approval process, as well as in diagnosis of conditions associated with alterations in RR activity

Alternatively, the methods of the present invention find use in detecting injuries due to exposure to toxic environmental chemicals such as industrial and occupational chemicals, environmental pollutants, pesticides, food additives, cosmetics, and the like.

### BRIEF DESCRIPTION OF THE DRAWINGS

FIG. 1 is a depiction of the de novo synthesis pathway of DNA. FIG. 1A shows the pathway as it occurs under normal conditions. FIG. 1B is a schematic of nucleoside fluxes in response to RR inhibition. A unique feature of RR is the response of nucleoside metabolism to its inhibition. When RR is inhibited, the activity of thymidine kinase is increased. This results in increased salvage of thymidine and reduced relative contribution from de novo thymidine synthesis into DNA (see text for further details).

FIG. 2 depicts the deoxyribose (dR) moiety of dNTPs in replicating DNA labeled endogenously, through the de novo nucleotide synthesis pathway, using stable isotope-labeled glucose or deuterated water (² H₂O).

FIG. 3 is a comparison of cell proliferation measured by the methods of the present invention with a standard cell counting technique in hydroxyurea-treated MCF7 cells. The graph depicts a strong correlation between the two methods.

FIG. 4 shows RR activity measured by ²H₂O in hydroxyurea-treated MCF7 cells. RR activity is dramatically inhibited at doses 50 μM HU and above.

FIG. 5 shows cell proliferation and RR activity measured by ²H₂O in hydroxyurea-treated SW1573 cells. Hydroxyurea inhibits RR activity by 80% at the low dose (6 μM).

FIG. 6 shows cell proliferation and RR activity measured by ²H₂O in gemcitabine-treated SW1573 cells. Gemcitabine inhibits RR activity by 70% at 1.6 nM and nearly abolishes it at 48 nM.

FIG. 7 depicts a simultaneous determination of RR inhibition in response to chemotherapy in bone marrow and tumor.

FIG. 8 shows in vivo tumor cell proliferation over 5 days of chemotherapy treatment (gemcitabine and hydroxyurea).

FIG. 9 depicts tumor volume over 5 days of chemotherapy treatment (gemzar and hydroxyurea). Significantly, no change in tumor volume was observed whereas changes in cell proliferation and RR activity (i.e., RR inhibition) were observed by the methods of the present invention over the same time course.

FIG. 10 is a schematic diagram showing the drug discovery, development, and approval (DDDA) process using effects on RR (i.e., data collected by the methods of the present invention) as a means for deciding to continue or cease efforts.

FIG. 11 illustrates use of the inventions herein in a drug discovery process.

### DETAILED DESCRIPTION OF THE INVENTION

I. Introduction

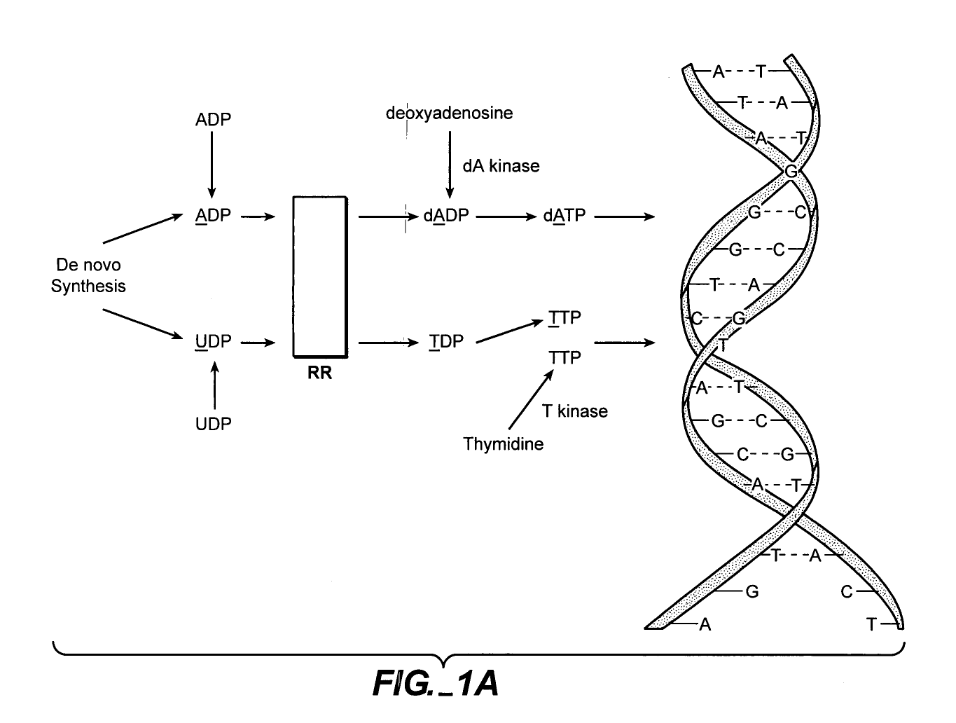

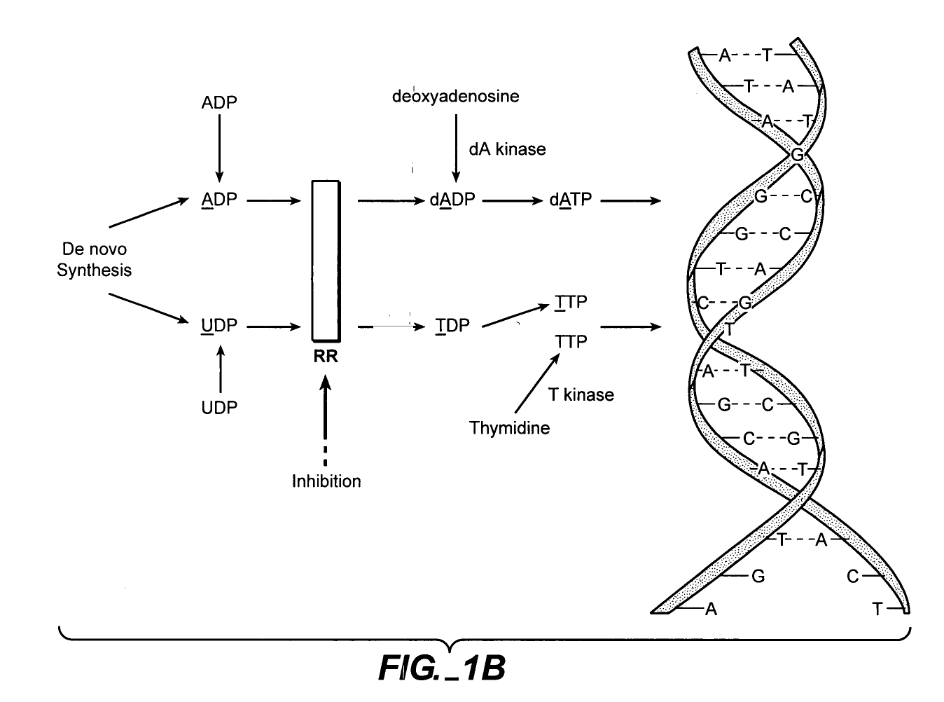

Ribonucleotide reductase (RR) catalyzes the conversion of ribonucleotides to deoxyribonucleotides (FIG. 1). A unique feature of RR is the response of nucleoside metabolism to its inhibition. When RR is inhibited, the activity of thymidine kinase (tk) is increased. This results in increased salvage of thymidine and reduced relative contribution from de novo thymidine synthesis into DNA. Traditionally, this effect is assayed in vitro by measuring the increase in the concentration of the free (thymidine triphosphate (TTP) pool relative to other deoxynucleotides. In contrast to tk, basal deoxyadenosine kinase (dAk) activity is low (Krygier V., J Biol Chem. 246:2752-2757 (1971), hereby incorporated by reference in its entirety, and in particular for the assay of dAk activity) and is not up-regulated in response to RR inhibition. The salvage of deoxyadenosine (dA) is very low in normal cells allowing for the measurement of DNA synthesis from the incorporation of de novo deoxyadenosine; furthermore, since dAk activity is not affected by RR inhibition it can be used as the denominator for calculating fractional de novo thymidine synthesis (see, infra). The relative inhibition of RR can be inferred from the reduction in de novo thymidine incorporation relative to dA incorporation into DNA.

RR is the target of several commonly used chemotherapies, including gemcitabine (Gem) and hydroxyurea (HU). In spite of its prominent role in DNA synthesis and as a chemotherapy target, there are no simple, quantitative in vivo assays to measure RR activity. The present invention utilizes previous methods measuring DNA synthesis as an indicator of cell proliferation, in vivo, using ²H₂O (U.S. Pat. No. 5,910,403, herein incorporated by reference). This methodology is based on the constitutive and predominant contribution from de novo synthesized dA to new DNA synthesis. Unlike thymidine and deoxycytosine (dC), which are actively salvaged and incorporated into DNA, the salvage of dA is small. The in vivo salvage of thymidine has long been exploited for determining cell proliferation (e.g., Brdu and ³H-thymidine incorporation). Because of the variable salvage of thymidine, these latter methods are of limited value for quantitative measurements.

In the present invention, the 2H₂O labeling technique for measurement of DNA synthesis (U.S. Pat. No. 5,910,403, supra) is adapted to include the measurement of RR modulation (particularly inhibition) and thymidine salvage. This is achieved by comparing the differential incorporation of a label into pyrimidines (e.g. dA) versus purines (e.g. T); for example, one embodiment of the invention utilizes a comparison of the ²H incorporation in dA to the ²H incorporation into T in DNA. Previous studies have demonstrated (Takahashi et al. Cancer Chemother. Pharmacol. 41: 268-274) that inhibition of RR results in up regulation of the salvage of pyrimidine nucleosides (e.g., dC kinase and T kinase) but not dA. Accordingly, the present invention capitalizes on the fact that the dilution of T relative to dA reflects inhibition of RR, although as outlined below, the invention allows the determination of modulation of RR activity, including both increases (agonism) as well as decreases (antagonism)

The basic principles underlying the present invention are shown in FIG. 1. The methods of the present invention make use of the intrinsic regulatory architecture of nucleoside metabolic pathways (FIG. 1) to determine modulation (including inhibition or activation) of RR activityIncorporation of an endogenous label for dR, such as ²H₂O or ²H-glucose (FIG. 1) will be influenced by several factors:
a) Dilution of administered label in the tissue precursor pool, e.g., by unlabeled tissue water or free glucose;
b) Dilution of label in dR of PRPP (FIG. 1) by unlabeled metabolic precursors of dR, e.g., glycogenolysis, gluconeogenesis, pentose-phosphate pathway;
c) Dilution of label in dNTPs by input of unlabeled dNTPs through dN salvage pathways, e.g., thymidine kinase, dC kinase;
d) Dilution of label in DNA by unlabeled DNA in the population of cells, e.g, pre-existing cells that did not divide during the period of label administration.

The invention is based on the fact that inhibition of RR activity results in differential dilution of pyrimidine vs. purine dNTPs isolated from DNA due to differences at dilution step c, supra.

The difference between pyrimidine and purine label enrichment is based on differential regulation of pyrimidines and purines after the RR step: namely, upregulation of pyrimidine dN salvage but little or no input from purine dN salvage. Because all of the other potential dilution steps (a, b, and d, supra) are shared for purine and pyrimidine dNs isolated from DNA (FIG. 1), any differences at step c are apparent. The other steps cancel out because dilution in tissue, dilution of PRPP, and dilution by pre-existing cells will be identical for pyrimidine and purine dNs in DNA (FIG. 1). Specifically, the labeling ratio of dT/dA (or dT/dG, or dC/dA, or dC/dG) during administration of ²H₂O or ²H-glucose will reflect changes in RR activity relative to dN salvage.

In general, the invention can be practiced in a variety of different ways. In one embodiment, as is more fully outlined below, the administration of an isotope-labeled substrate into a living system (for example an individual with a disease, an individual that has been exposed to a drug, a cell line of interest, etc.) results in the incorporation of the label into purine and pyrimidine deoxynucleosides, resulting in the eventual conversion of this pool of labeled deoxynucleosides into DNA containing labeled deoxynucleotides. The ratio of labeled purine:pyrimidine (or vice versa) is a ratio that can be compared to other living systems, e.g control living systems without the disease or drug. Differential incorporation (and thus different ratios) are an indication of the modulation of the RR activity. This can be done as a sampling of the system at a single time point after administration of the isotope-labeled substrate (either as a continuous or discontinuous administration), or with multiple sampling at a plurality (e.g. two or more) time points. In addition, a single sampling can be done after a single administration, or single sampling can be done after administration for different times. For example, administration of a short bolus of label, followed by incubation and a sampling at time X can be done; the system is then allowed to clear, and a second administration of a longer bolus can be done, followed again by incubation and sampling. Alternatively, multiple samplings and multiple administrations can be done.

Alternatively, the amount of label incorporation can be used to calculate the molecular flux rate for the increase of the labeled dNs into DNA. This can also be done as outlined above using single sampling at a single time point (and calculating the rate based on the zero time point) or with multiple sampling, multiple time points or both. Ratios of molecular flux rates are then compared to elucidate alterations in RR activity.

II. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999), all of which are incorporated by reference for the needed techniques. Furthermore, procedures employing commercially available assay kits and reagents will typically be used according to manufacturer-defined protocols unless otherwise noted.

III. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999, hereby incorporated by reference in its entirety, and in particular for the techniques outlined therein). As appropriate, procedures involving the use of commercially available kits and reagents are generally carried out in accordance with manufacturer defined protocols and/or parameters unless otherwise noted.

“Molecular flux rates” refers to the rate of synthesis and/or breakdown of molecules within a cell, tissue, or organism. “Molecular flux rates” also refers to a molecule's input into or removal from a pool of molecules, and is therefore synonymous with the flow into and out of said pool of molecules.

“Metabolic pathway” refers to any linked series of two or more biochemical steps in a living system (i.e., a biochemical process), the net result of which is a chemical, spatial or physical transformation of a molecule or molecules. Metabolic pathways are defined by the direction and flow of molecules through the biochemical steps that comprise the pathway. Molecules within metabolic pathways can be of any biochemical class, e.g., including but not limited to lipids, proteins, amino acids, carbohydrates, nucleic acids, polynucleotides, porphyrins, glycosaminoglycans, glycolipids, intermediary metabolites, inorganic minerals, ions, etc.

“Flux rate through a metabolic pathway” refers to the rate of molecular transformations through a defined metabolic pathway. The unit of flux rates through pathways is chemical mass per time (e.g., moles per minute, grams per hour). Flux rate through a pathway optimally refers to the transformation rate from a clearly defined biochemical starting point to a clearly defined biochemical end-point, including all the stages in between in the defined metabolic pathway of interest.

“Isotopes” refer to atoms with the same number of protons and hence of the same element but with different numbers of neutrons (e.g., ¹H vs. ²H or D). The term “isotope” includes “stable isotopes”, e.g. non-radioactive isotopes, as well as “radioactive isotopes”, e.g. those that decay over time.

“Isotopologues” refer to isotopic homologues or molecular species that have identical elemental and chemical compositions but differ in isotopic content (e.g., CH₃NH₂ vs. CH₃NHD in the example above). Isotopologues are defined by their isotopic composition, therefore each isotopologue has a unique exact mass but may not have a unique structure. An isotopologue is usually comprised of a family of isotopic isomers (isotopomers) which differ by the location of the isotopes on the molecule (e.g., CH₃NHD and CH₂DNH₂ are the same isotopologue but are different isotopomers).

“Isotope-labeled water” includes water labeled with one or more specific heavy isotopes of either hydrogen or oxygen. Specific examples of isotope-labeled water include ²H₂O, ³H₂O, and H₂¹⁸O.

“Candidate agent” or “candidate drug” as used herein describes any molecule, e.g., proteins including biotherapeutics including antibodies and enzymes, small organic molecules including known drugs and drug candidates, polysaccharides, fatty acids, vaccines, nucleic acids, etc. that can be screened for activity as outlined herein. Candidate agents are evaluated in the present invention for discovering potential therapeutic agents that affect RR activity and therefore potential disease states, for elucidating toxic effects of agents (e.g. environmental pollutants including industrial chemicals, pesticides, herbicides, etc.), drugs and drug candidates, food additives, cosmetics, etc., as well as for elucidating new pathways associated with agents (e.g. research into the side effects of drugs, etc.).

Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

“Known drugs” or “known drug agents” or “already-approved drugs” refers to agents (i.e., chemical entities or biological factors) that have been approved for therapeutic use as drugs in human beings or animals in the United States or other jurisdictions. In the context of the present invention, the term “already-approved drug” means a drug having approval for an indication distinct from an indication being tested for by use of the methods disclosed herein. Using psoriasis and fluoxetine as an example, the methods of the present invention allow one to test fluoxetine, a drug approved by the FDA (and other jurisdictions) for the treatment of depression, for effects on biomarkers of psoriasis (e.g., keratinocyte proliferation or keratin synthesis); treating psoriasis with fluoxetine is an indication not approved by FDA or other jurisdictions. In this manner, one can find new uses (in this example, anti-psoriatic effects) for an already-approved drug (in this example, fluoxetine).

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations. Peptide inhibitors of enzymes find particular use.

In a preferred embodiment, the candidate bioactive agents are naturally occuring proteins or fragments of naturally occuring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

In a preferred embodiment, the candidate agents are antibodies, a class of proteins. The term “antibody” includes full-length as well antibody fragments, as are known in the art, including Fab Fab2, single chain antibodies (Fv for example), chimeric antibodies, humanized and human antibodies, etc., either produced by the modification of whole antibodies or those synthesized de novo using recombinant DNA technologies, and derivatives thereof.

In a preferred embodiment, the candidate bioactive agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occuring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.etc.

It should be noted in the context of the invention that nucleosides (ribose plus base) and nucleotides (ribose, base and at least one phosphate) are used interchangeably herein unless otherwise noted. The label is generally incorporated into the ribose, although RR catalyzes the conversion of ribonucleotides to deoxyribonucleotides (also referred to herein as “deoxynucleosides/tides”).

As described above generally for proteins, nucleic acid candidate bioactive agents may be naturally occuring nucleic acids, random and/or synthetic nucleic acids. For example, digests of procaryotic or eucaryotic genomes may be used as is outlined above for proteins. In addition, RNAis are included herein.

“Food additive” includes, but is not limited to, organoleptic agents (i.e., those agents conferring flavor, texture, aroma, and color), preservatives such as nitrosamines, nitrosamides, N-nitroso substances and the like, congealants, emulsifiers, dispersants, fumigants, humectants, oxidizing and reducing agents, propellants, sequestrants, solvents, surface-acting agents, surface-finishing agents, synergists, pesticides, chlorinated organic compounds, any chemical ingested by a food animal or taken up by a food plant, and any chemical leaching into (or otherwise finding its way into) food or drink from packaging material. The term is meant to encompass those chemicals which are added into food or drink products at some step in the manufacturing and packaging process, or find their way into food by ingestion by food animals or uptake by food plants, or through microbial byproducts such as endotoxins and exotoxins (pre-formed toxins such as botulinin toxin or aflatoxin), or through the cooking process (such as heterocyclic amines, e.g., 2-amino-3-methyllimidazo[4,5-f]quinolone), or by leaching or some other process from packaging material during manufacturing, packaging, storage, and handling activities.

“Industrial chemical” includes, but is not limited to, volatile organic compounds, semi-volatile organic compounds, cleaners, solvents, thinners, mixers, metallic compounds, metals, organometals, metalloids, substituted and non-substituted aliphatic and acyclic hydrocarbons such as hexane, substituted and non-substituted aromatic hydrocarbons such as benzene and styrene, halogenated hydrocarbons such as vinyl chloride, aminoderivatives and nitroderivatives such as nitrobenzene, glycols and derivatives such as propylene glycol, ketones such as cyclohexanone, aldehydes such as furfural, amides and anhydrides such as acrylamide, phenols, cyanides and nitriles, isocyanates, and pesticides, herbicides, rodenticides, and fungicides.

“Environmental pollutant” includes any chemical not found in nature or chemicals that are found in nature but artificially concentrated to levels exceeding those found in nature (at least found in accessible media in nature). So, for example, environmental pollutants can include any of the non-natural chemicals identified as an occupational or industrial chemical yet found in a non-occupational or industrial setting such as a park, school, or playground. Alternatively, environmental pollutants may comprise naturally occurring chemicals such as lead but at levels exceeding background (for example, lead found in the soil along highways deposited by the exhaust from the burning of leaded gasoline in automobiles). Environmental pollutants may be from a point source such as a factory smokestack or industrial liquid discharge into surface or groundwater, or from a non-point source such as the exhaust from cars traveling along a highway, the diesel exhaust (and all that it contains) from buses traveling along city streets, or pesticides deposited in soil from airborne dust originating in farmlands. As used herein, “environmental contaminant” is synonymous with “environmental pollutant.”

“Living system” includes, but is not limited to, cells (including primary cells), cell lines (including cell lines of healthy and diseased cells), plants and animals, particularly mammals and particularly human. Suitable cells include, but are not limited to, tumor cells of all types (particularly melanoma, myeloid leukemia, carcinomas of the lung, breast, ovaries, colon, kidney, prostate, brain, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, myocytes, fibroblasts, neurons, glial cells, pancreatic cells, intestinal epithelial cells, lymphocytes, erythrocytes, microbial cells and any other cell-type that can be maintained alive and functional in vitro. Microbial and plant cells can also be used.

In one embodiment, the cells may be genetically engineered, that is, contain exogeneous nucleic acid.

The cell may be collected from a multicellular organism and cultured or may be purchased from a commercial source such as the American Type Culture Collection and propagated as a cell line using techniques well known in the art. Suitable cell lines include, but are not limited to, cell lines made from any of the above-mentioned cells, as well as established cell lines such as Suitable cells also include known research cells, including, but not limited to, Jurkat T cells, NIH3T3 cells, CHO, Cos, etc. See the ATCC cell line catalog, hereby expressly incorporated by reference. Suitable mammals include, but are not limited to, any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are included within the definition herein. Living systems can either be control systems, which are free from perturbation such as treatment with candidate agents or free of disease or risk of disease, or systems under evaluation. “Living system” includes individual subjects, including human patients.

“Excreta” is defined herein as any liquid, solid, or gaseous material that is released from the body to the outside world. Examples include, but are not limited to, stool (feces), urine, seminal fluid, sputum, breast ductal fluid, saliva, cervical secretions, vaginal secretions, skin scrapings, skin flakes, hair, nasal secretions, pancreatic-biliary secretions, lacrimal fluid (tears), sweat, flatus, exhaled respiratory gases, or other physical materials released from the body.

A “biological sample” encompasses any sample obtained from a living system, including cells, tissues, or an organism. The sample may be solid in nature. The definition also encompasses liquid samples of biological origin, that are accessible from an organism through sampling by minimally invasive or non-invasive approaches (e.g., urine collection, needle aspiration, breast fluid collection from breast ductal lavage, skin scraping, semen collection, vaginal secretion collection, nasal secretion collection, sputum collection, stool collection, and other procedures involving minimal risk, discomfort or effort). The definition also includes samples that have been manipulated in any way after their procurement, such as by treatment with reagents, solubilization, or enrichment for certain components, such as proteins, lipids, carbohydrates, or organic metabolites. The term “biological sample” also encompasses a clinical sample such as biological fluid or tissue sample.

By “body spaces in communication with the external environment” is meant any space having contact with any media of the external environment. This contact may not be direct, but may in fact be indirect with contact by way of a duct or other body cavity ultimately having direct contact with any media of the external environment. For example, intestinal epithelial cells sloughed off from the gastrointestinal (GI) epithelial membrane are in a body space in communication with the external environment when they are located within, or associated with, the lumen of the gastrointestinal tract. Similarly, any intestinal epithelial molecules located within the lumen of the GI tract are also located within a body space in communication with the external environment. Skin cells (keratinocytes) residing on or associated with the surface of the epidermis are also in a body space in communication with the external environment. Furthermore, breast epithelial cells contained within breast fluid within a breast duct are in a body space in communication with the external environment as are prostate epithelial cells contained within seminal fluid found within the ductal system of the male reproductive and sex organs. Further examples include, but are not limited to: cervical epithelial cells within the vagina; vaginal epithelial cells within the vagina; colon epithelial cells (colonocytes) within or associated with stool; endometrial cells within the uterus; bladder epithelial cells located within or associated with the lumen of the bladder or are contained within urine collected outside the body; and bronchial epithelial cells located within, or associated with, the lumen of the airways (e.g., bronchi, bronchioli, alveolar sacs).

“Biological fluid” refers to, but is not limited to, urine, edema fluid, saliva, lacrimal fluid, inflammatory exudates, synovial fluid, abscess, empyema or other infected fluid, sweat, pulmonary secretions (sputum), seminal fluid, feces, bile, intestinal secretions, vaginal secretions, or any other biological fluid found in spaces external to the body (i.e., luminal or integumentary spaces).

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Mass isotopomer” refers to family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nominal mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as MO; for most organic molecules, this is the species containing all ¹²C, ¹H, ¹⁶O, ¹⁴N, etc. Other mass isotopomers are distinguished by their mass differences from MO (M₁, M₂, etc.). For a given mass isotopomer, the location or position of isotopes within the molecule is not specified and may vary (i.e., “positional isotopomers” are not distinguished).

“Mass isotopomer envelope” refers to the set of mass isotopomers comprising the family associated with each molecule or ion fragment monitored.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonomously with the term “mass isotopomer pattern.”

“Monoisotopic mass” refers to the exact mass of the molecular species that contains all ¹ H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m₀ and the masses of other mass isotopomers are identified by their mass differences from m₀ (m₁, m₂, etc.).

“Isotopically perturbed” refers to the state of an element or molecule that results from the explicit incorporation of an element or molecule with a distribution of isotopes that differs from the distribution that is most commonly found in nature, whether a naturally less abundant isotope is present in excess (enriched) or in deficit (depleted). Thus the labels of the present invention are isotopically perturbed, as is the DNA into which the labels are incorporated.

“Metabolic precursors” or “precursors” refer to molecules or atoms that enter into molecular end-products of interest through the metabolic processes of the cell or organism (i.e., through biosynthetic, degradative, and/or intermediary metabolic pathways).

By “molecule of interest” is meant any molecule (polymer and/or monomer), including but not limited to, amino acids, carbohydrates, fatty acids, peptides, sugars, lipids, nucleic acids, polynucleotides, glycosaminoglycans, polypeptides, or proteins that are present within a metabolic pathway within a cell. In the context of the present invention, a “molecule of interest” may be a “biomarker” of an disease and its flux rate, relative to the flux rate of an unexposed or otherwise healthy subject (i.e., control subject), may represent clinically non-observant or subtle pathophysiological occurrences in a subject of interest that may be predictive of future disease or injury in the subject. In this manner, comparing the flux rates of one or more biomarkers of interest in a subject with the flux rates of one or more biomarkers of interest in a control subject, will find use in diagnosing the subject with, or evaluating or quantifying the subject's risk in acquiring, an disease of interest. Moreover, such information will find use in establishing a prognosis for a subject having an disease of interest, monitoring the progression of an disease of interest in a subject, or evaluating the therapeutic efficacy of a treatment regimen in a subject having an disease of interest.

“Monomer” refers to a chemical unit that combines during the synthesis of a polymer and which is present two or more times in the polymer.

“Polymer” refers to a molecule synthesized from and containing two or more repeats of a monomer. Polymers may be homopolymers (all monomers identical) or heteropolymers (more than one type of monomer). A “biopolymer” is a polymer synthesized by or in a living system or otherwise associated with a living system.

By “DNA” is meant a polymeric form of deoxyribonucleotides (adenine, guanine, thymine, or cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. The term captures molecules that include the four bases adenine, guanine, thymine, or cytosine, as well as molecules that include base analogs which are known in the art.

“Isotope labeled substrate” includes any isotope-labeled precursor molecule that is able to be incorporated into DNA (or the deoxyribonucleotide moiety that DNA is comprised of) in a living system. Examples of isotope labeled substrates include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃.

“Deuterated water” refers to water incorporating one or more ²H isotopes.

“Labeled glucose” refers to glucose labeled with one or more ²H isotopes. Specific examples of labeled glucose or ²H-labeled glucose include [6,6-²H₂]glucose, [1-²H₁]glucose, and [1,2,3,4,5,6-²H₇] glucose.

“Administer[ed]” includes a living system exposed to a compound, including candidate agents and labeled substrates, Such exposure can be from, but is not limited to, topical application, oral ingestion, inhalation, subcutaneous injection, intraperitoneal injection, intravenous injection, and intraarterial injection, in animals or other higher organisms. Administration to cells, tissue culture or cell lines can be adding the compound to the growth media.

By “toxic effect” is meant an adverse response by a living system to a chemical entity or known drug agent. A toxic effect can be comprised of, for example, end-organ toxicity.

“At least partially identified” in the context of drug discovery and development means at least one clinically relevant pharmacological characteristic of a drug agent (i.e., a “compound”) has been identified using one or more of the methods of the present invention. This characteristic may be a desirable one, for example, increasing or decreasing molecular flux rates through a metabolic pathway that contributes to a disease process, altering signal transduction pathways or cell surface receptors that alter the activity of metabolic pathways relevant to a disease, inhibiting activation of an enzyme and the like. Alternatively, a pharmacological characteristic of a drug agent may be an undesirable one for example, the production of one or more toxic effects. There are a plethora of desirable and undesirable characteristics of drug agents well known to those skilled in the art and each will be viewed in the context of the particular drug agent being developed and the targeted disease. Of course, a drug agent can be more than at least partially identified when, for example, when several characteristics have been identified (desirable or undesirable or both) that are sufficient to support a particular milestone decision point along the drug development pathway. Such milestones include, but are not limited to, pre-clinical decisions for in vitro to in vivo transition, pre-IND filing go/no go decision, phase I to phase II transition, phase II to phase III transition, NDA filing, and FDA approval for marketing. Therefore, “at least partially” identified includes the identification of one or more pharmacological characteristics useful in evaluating a drug agent in the drug discovery/drug development process. A pharmacologist or physician or other researcher may evaluate all or a portion of the identified desirable and undesirable characteristics of a drug agent to establish its therapeutic index. This may be accomplished using procedures well known in the art.

“Manufacturing a drug agent” in the context of the present invention includes any means, well known to those skilled in the art, employed for the making of a drug agent product. Manufacturing processes include, but are not limited to, medicinal chemical synthesis (i.e., synthetic organic chemistry), combinatorial chemistry, biotechnology methods such as hybridoma monoclonal antibody production, recombinant DNA technology, and other techniques well known to the skilled artisan. Such a product may be a final drug agent that is marketed for therapeutic use, a component of a combination product that is marketed for therapeutic use, or any intermediate product used in the development of the final drug agent product, whether as part of a combination product or a single product. “Manufacturing drug agent” is synonymous with “manufacturing a compound.”

By “action” is meant a specific and direct consequence of an intervention such as the administering of a drug.

By “therapeutic action” is meant an effect on a biochemical or molecular process (i.e., the flow of molecules through metabolic pathways or networks) in a manner that is beneficial to the organism. The effect may be responsible for, or contributing in, a causal manner to the initiation, progression, severity, pathology, aggressiveness, grade, activity, disability, mortality, morbidity, disease sub-classification or other underlying pathogenic or pathologic feature of one or more diseases wherein said effect is beneficial to health or otherwise contributes to a desirable outcome (e.g., a desirable clinical outcome).

By “biomarker” is meant a physical, biochemical, or physiologic measurement from or on the organism that represents a true or intended mechanistic target of a compound or a mechanistic event believed to be responsible for, or contributing in, a causal manner to the initiation, progression, severity, pathology, aggressiveness, grade, activity, disability, mortality, morbidity, disease sub-classification or other underlying pathogenic or pathologic feature of one or more diseases. In some embodiments, there may be a correlational effect instead of a causal one. A biomarker may be the target for monitoring the outcome of a therapeutic intervention (i.e., the functional or structural target of a drug agent). As defined herein “biomarker” refers to biochemical processes that are involved in, or are believed to be involved in, the etiology or progression of a disease or disorder. The biochemical process (i.e., the flow of molecules through a targeted metabolic pathway or network) is the focus of analysis (as disclosed herein) since it is the underlying changes of the biochemical process (i.e., molecular flux rates) that may be the significant or authentic target for treatment or diagnostic monitoring of the disease or disorder.

By “evaluate” or “evaluation” or “evaluating,” in the context of the present invention, is meant a process whereby the activity, toxicity, relative potency, potential therapeutic value and/or efficacy, significance, or worth of a chemical entity, biological factor, combination of chemical entities, or combination of biological factors is determined through appraisal and study, usually by means of comparing experimental outcomes to established standards and/or conditions. The term embraces the concept of providing sufficient information for a decision-maker to make a “go/no go” decision on a chemical entity or biological factor (or combinations of chemical entities or combinations of biological factors) to proceed further in the drug development process. A “go/no go” decision may be made at any point or milestone in the drug development process including, but not limited to, any stage within pre-clinical development, the pre-clinical to Investigational New Drug (IND) stage, the Phase I to Phase II stage, the Phase II to more advanced phases within Phase II (such as Phase IIb), the Phase II to Phase III stage, the Phase III to the New Drug Application (NDA) or Biologics License Application (BLA) stage, or stages beyond (such as Phase IV or other post-NDA or post-BLA stages). The term also embraces the concept of providing sufficient information to select “best-in-breed” (or “best-of-breed”) in a class of compounds (chemical entities, biologics).

By “characterize,” “characterizing,” or “characterization,” in the context of the present invention is meant an effort to describe the character or quality of a chemical entity or biological factor or combination of chemical entities or combination of biological factors (or mixtures thereof). As used herein, the term is nearly equivalent to “evaluate,” yet lacks the more refined aspects of “evaluate,” in which to “evaluate” a drug includes the ability to make a “go/no go” decision (based on an assessment of therapeutic value) on proceeding with that drug or chemical entity or biological factor through the drug development process.

By “condition” or “medical condition” is meant the physical status of the body as a whole or of one of its parts. The term is usually used to indicate a change from a previous physical or mental status, or an abnormality not recognized by medical authorities as a disease or disorder. Examples of “conditions” or “medical conditions” include, but are not limited to, obesity, cancer, proliferative diseases and pregnancy.

By “therapeutic effect” is meant any effect elicited by a compound or combination of compounds or mixtures of compounds that provides ameliorative or palliative results, or improves, even to the slightest degree, any clinical sign or symptom of a disease or condition.

IV. Methods of the Invention

A. Overview of the Methods of the Invention

The present invention is directed to methods of measuring cell proliferation by means of measuring de novo DNA synthesis and to methods for assessing ribonucleotide reductase activity (i.e., RR inhibition) by comparing the incorporation of purine and pyrimidine deoxynucleosides such as deoxyadenosine and deoxythymidine into DNA. While the methods below generally center on the use of deoxyadenosine and deoxythymidine, other deoxynucleosides can be used. The methods of the present invention allow for the measurement of cell proliferation and RR activity simultaneously. Salvage of deoxyadenosine is very low in normal cells—this fact allows for the measurement of DNA synthesis from the incorporation of de novo deoxyadenosine; furthermore, since dAk activity is not affected by RR inhibition it can be used as the denominator for calculating fractional de novo thymidine synthesis. The relative inhibition of RR can be inferred from the reduction in de novo thymidine incorporation relative to dA into DNA.

B. Administering Isotope-Labeled Substrates

As a first step in the methods of the invention, isotope-labeled substrates are administered. These substrates are generally metabolic precursors, e.g. they are taken up within the living system and enzymatically converted; in the present invention, the substrates are converted to deoxynucleosides which are then incorporated into DNA.

1 . Administering an Isotope-Labeled Substrate Molecule

Modes of administering the one or more isotope-labeled substrates may vary, depending upon the absorptive properties of the isotope-labeled substrate and the specific biosynthetic pool into which each compound is targeted. Precursors may be administered to organisms, plants and animals including humans directly for in vivo analysis. In addition, precursors may be administered in vitro to living cells.

Generally, an appropriate mode of administration is one that produces a steady state level of precursor within the biosynthetic pool and/or in a reservoir supplying such a pool for at least a transient period of time. Intravascular or oral routes of administration are commonly used to administer such precursors to organisms, including humans. Other routes of administration, such as subcutaneous or intramuscular administration, optionally when used in conjunction with slow release substrate compositions, are also appropriate. Compositions for injection are generally prepared in sterile pharmaceutical excipients.

As is discussed herein, administration can be done continuously (e.g. up to and/or including the time of sampling) or discontinuously (either as a single dose over time or multiple doses). When discontinuous administration is done, the time of the individual administrations can either be the same or different.

a. Labeled Substrates

(1) Isotope Labels

The first step in measuring molecular flux rates involves administering an isotope-labeled substrate to a living system. The isotope labeled substrate may contain a stable isotope or a radioisotope. Isotope labels that can be used in accordance with the methods of the present invention include, but are not limited to, ²H, ¹³C, ¹⁵N, ⁸¹O, ³H, ¹⁴C, ³⁵S, ³²P, ³³P, ¹²⁵I, ¹³¹I, or other isotopes of elements present in organic systems. These isotopes, and others, are suitable for all classes of substrates (e.g., precursor molecules) envisioned for use in the present invention. Such precursor molecules include, but are not limited to, nucleic acid precursors,

In one embodiment, the isotope label is ²H.

i. Precursors of Nucleic Acids

Precursors of nucleic acids (i.e., RNA, DNA) are any compounds suitable for incorporation into RNA and/or DNA synthetic pathways. Examples of substrates useful in labeling the deoxyribose ring of DNA include, but are not limited to, [6,6-²H₂] glucose,[U-¹³C₆] glucose and [2-¹³C₁] glycerol (see U.S. Pat. No. 6,461,806, herein incorporated by reference in its entirety). Labeling of the deoxyribose is superior to labeling of the information-carrying nitrogen bases in DNA because it avoids variable dilution sources. The stable isotope labels are readily detectable by mass spectrometric techniques.

In one embodiment, a stable isotope label is used to label the deoxyribose ring of DNA from glucose, precursors of glucose-6-phosphate or precursors of ribose-5-phosphate. In embodiments where glucose is used as the starting material, suitable labels include, but are not limited to, deuterium-labeled glucose such as [6,6-²H₂] glucose, [1-²H₁] glucose, [3-²H₁] glucose, [²H₇] glucose, and the like; ¹³C-1 labeled glucose such as [1-¹³C₁] glucose, [U-¹³C₆] glucose and the like; and ¹⁸O-labeled glucose such as [1-¹⁸O₂] glucose and the like.

In embodiments where a glucose-6-phosphate precursor or a ribose-5-phosphate precursor is desired, a gluconeogenic precursor or a metabolite capable of being converted to glucose-6-phosphate or ribose-5-phosphate can be used. Gluconeogenic precursors include, but are not limited to, ¹³C-labeled glycerol such as [2-¹³C₁] glycerol and the like, a ¹³C-labeled amino acid, deuterated water (²H₂O) and ¹³C-labeled lactate, alanine, pyruvate, propionate or other non-amino acid precursors for gluconeogenesis. Metabolites which are converted to glucose-6-phosphate or ribose-5-phosphate include, but are not limited to, labeled (²H or ¹³C) hexoses such as [1-²H₁] galactose, [U-¹³C] fructose and the like; labeled (²H or ¹³C) pentoses such as [1-¹³C₁] ribose, [1-²H₁] xylitol and the like, labeled (²H or ¹³C) pentose phosphate pathway metabolites such as [1-²H₁] seduheptalose and the like, and labeled (²H or ¹³C) amino sugars such as [U-¹³C] glucosamine, [1-²H₁] N-acetyl-glucosamine and the like.

The present invention also encompasses stable isotope labels which label purine and pyrimidine bases of DNA through the de novo nucleotide synthesis pathway. Various building blocks for endogenous purine synthesis can be used to label purines and they include, but are not limited to, ¹⁵N-labeled amino acids such as [¹⁵N] glycine, [¹⁵N] glutamine, [¹⁵N] aspartate and the like, ¹³C-labeled precursors such as [1-¹³C₁] glycone, [3-¹³C₁] acetate, [¹³C]HCO₃, [¹³C] methionine and the like, and H-labeled precursors such as ²H₂O. Various building blocks for endogenous pyrimidine synthesis can be used to label pyrimidines and they include, but are not limited to, ¹⁵N-labeled amino acids such as [¹⁵N] glutamine and the like, ¹³C-labeled precursors such as [¹³C]HCO₃, [U-¹³C₄] aspartate and the like, and ²H-labeled precursors (²H₂O).

It is understood by those skilled in the art that in addition to the list above, other stable isotope labels which are substrates or precursors for any pathways which result in endogenous labeling of DNA are also encompassed within the scope of the invention. The labels suitable for use in the present invention are generally commercially available or can be synthesized by methods well known in the art.

ii. Water as a Precursor Molecule

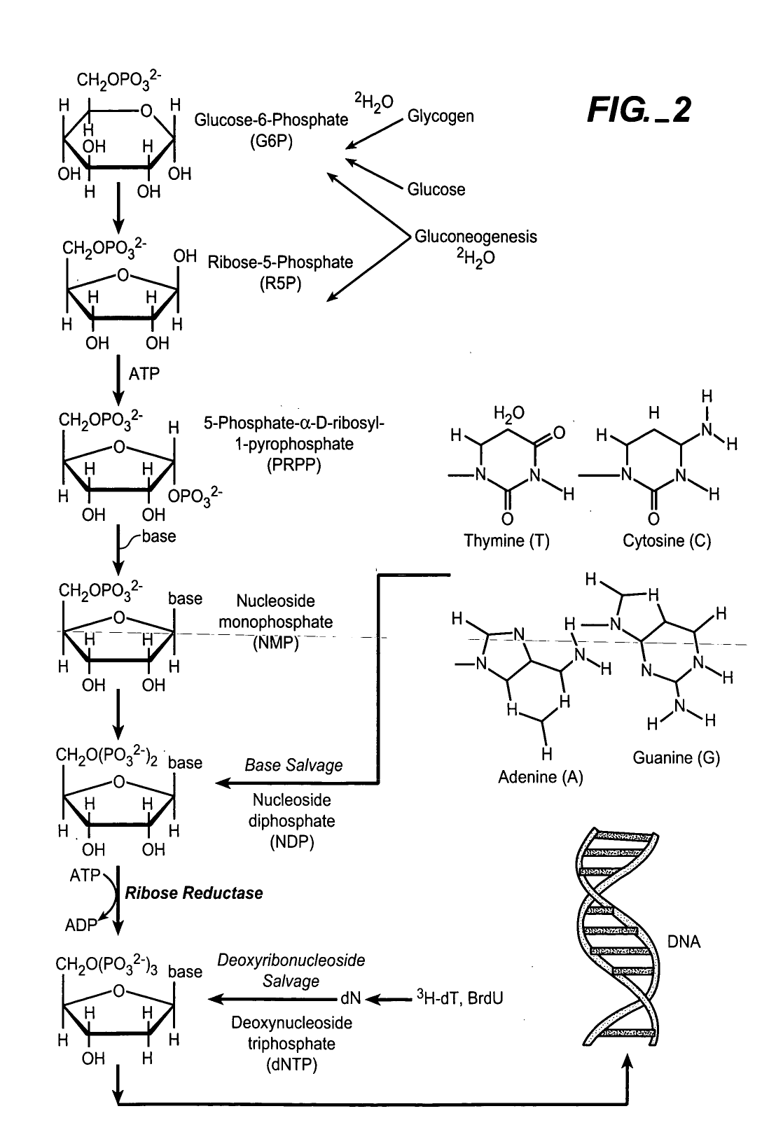

Water is a precursor of nucleic acids (see FIG. 2 and U.S. Provisional Patent Application Nos. 60/484,626 and 60/581,028, which are herein incorporated by reference in their entirety). As such, labeled water may serve as a precursor in the methods taught herein.

H₂O availability is probably never limiting for biosynthetic reactions in a cell (because H₂O represents close to 70% of the content of cells, or >35 Molar concentration), but hydrogen and oxygen atoms from H₂O contribute stochiometrically to many reactions involved in biosynthetic pathways:
e.g.,: R—CO—CH2—COOH+NADPH+H₂O →R—CH₂CH2COOH (fatty acid synthesis).

As a consequence, isotope labels provided in the form of H- or O-isotope-labeled water is incorporated into biological molecules as part of synthetic pathways. Hydrogen incorporation can occur in two ways: into labile positions in a molecule (i.e., rapidly exchangeable, not requiring enzyme catalyzed reactions) or into stable positions (i.e., not rapidly exchangeable, requiring enzyme catalysis). Oxygen incorporation occurs in stable positions.

Some of the hydrogen-incorporating steps from cellular water into C—H bonds in biological molecules only occur during well-defined enzyme-catalyzed steps in the biosynthetic reaction sequence, and are not labile (exchangeable with solvent water in the tissue) once present in the mature end-product molecules. For example, the C—H bonds on glucose are not exchangeable in solution. In contrast, each of the following C—H positions exchanges with body water during reversal of specific enzymatic reactions: C-1 and C-6, in the oxaloacetate/succinate sequence in the Krebs' cycle and in the lactate/pyruvate reaction; C-2, in the glucose-6-phosphate/fructose-6-phosphate reaction; C-3 and C-4, in the glyceraldehyde-3-phosphate/dihydroxyacetone-phosphate reaction; C-5, in the 3-phosphoglycerate/glyceraldehyde-3-phosphate and glucose-6-phosphate/fructose-6-phosphate reactions.

Labeled hydrogen or oxygen atoms from water that are covalently incorporated into specific non-labile positions of a molecule thereby reveals the molecule's “biosynthetic history”—i.e., label incorporation signifies that the molecule was synthesized during the period that isotope-labeled water was present in cellular water.

The labile hydrogens (non-covalently associated or present in exchangeable covalent bonds) in these biological molecules do not reveal the molecule's biosynthetic history. Labile hydrogen atoms can be easily removed by incubation with unlabelled water (H₂O) (i.e., by reversal of the same non-enzymatic exchange reactions through which ²H or ³H was incorporated in the first place), however:

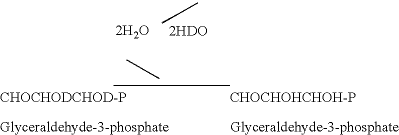

As a consequence, potentially contaminating hydrogen label that does not reflect biosynthetic history, but is incorporated via non-synthetic exchange reactions, can easily be removed in practice by incubation with natural abundance H₂O.

Analytic methods are available for measuring quantitatively the incorporation of labeled hydrogen atoms into biological molecules (e.g., liquid scintillation counting for ³H; mass spectrometry or NMR spectroscopy for ²H and ¹⁸O). For further discussions on the theory of isotope-labeled water incorporation, see, for example, Jungas R L. Biochemistry. 1968 7:3708-17, incorporated herein by reference.

Labeled water may be readily obtained commercially. For example, H₂O may be purchased from Cambridge Isotope Labs (Andover, Mass.), and ³H₂O may be purchased, e.g., from New England Nuclear, Inc. In general, ²H₂O is non-radioactive and thus, presents fewer toxicity concerns than radioactive ³H₂O. ²H₂O may be administered, for example, as a percent of total body water, e.g., 1% of total body water consumed (e.g., for 3 liters water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved relatively inexpensively using the techniques of the invention. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of 2H₂O. The Applicant has discovered that as long as rapid changes in body water enrichment are prevented (e.g., by initial administration in small, divided doses), high body water enrichments of ²H₂O can be maintained with no toxicities. For example, the low expense of commercially available ²H₂O allows long-term maintenance of enrichments in the 1-5% range at relatively low expense.

Relatively high and relatively constant body water enrichments for administration of H₂¹⁸O may also be accomplished, since the ¹⁸O isotope is not toxic, and does not present a significant health risk as a result.

Isotope-labeled water may be administered via continuous isotope-labeled water administration, discontinuous isotope-labeled water administration, or after single or multiple administration of isotope-labeled water administration. In continuous isotope-labeled water administration, isotope-labeled water is administered to an individual for a period of time sufficient to maintain relatively constant water enrichments over time in the individual. For continuous methods, labeled water is optimally administered for a period of sufficient duration to achieve a steady state concentration (e.g., 3-8 weeks in humans, 1-2 weeks in rodents).

In discontinuous isotope-labeled water administration, an amount of isotope-labeled water is measured and then administered, one or more times, and then the exposure to isotope-labeled water is discontinued and wash-out of isotope-labeled water from body water pool is allowed to occur. The time course of delabeling may then be monitored. Water is optimally administered for a period of sufficient duration to achieve detectable levels in biological molecules.

Isotope-labeled water may be administered to an individual or tissue or cells in various ways known in the art. For example, isotope-labeled water may be administered orally, parenterally, subcutaneously, intravascularly (e.g., intravenously, intraarterially), or intraperitoneally. Several commercial sources of ²H₂O and H₂¹⁸O are available, including Isotec, Inc. (Miamisburg Ohio, and Cambridge Isotopes, Inc. (Andover, Mass.). The isotopic content of isotope labeled water that is administered can range from about 0.001% to about 20% and depends upon the analytic sensitivity of the instrument used to measure the isotopic content of the biological molecules. In one embodiment, 4% ²H₂O in drinking water is orally administered. In another embodiment, a human is administered 50 mL of ²H₂O orally.

The living system being administered 2H₂O may be a cell. The cell may be collected from a multicellular organism and cultured or may be purchased from a commercial source such as the American Type Culture Collection and propagated as a cell line using techniques well known in the art. Alternatively, the individual being administered ²H₂O may be any multicellular organism including a mammal.

In variations involving the administering of drugs, drug candidates, drug leads, biological factors, or combinations thereof (i.e., compounds, combinations of compounds, or mixtures of compounds), the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

C. Administration of Candidate Agents

As outlined herein, candidate agents are administered for a variety of reasons. In some embodiments, the candidate agents are evaluated in the present invention for discovering potential therapeutic agents that affect RR activity and therefore potential disease states, for elucidating toxic effects of agents (e.g. environmental pollutants including industrial chemicals, pesticides, herbicides, etc.), drugs and drug candidates, food additives, cosmetics, etc., as well as for elucidating new pathways associated with agents (e.g. research into the side effects of drugs, etc.). Administration is accomplished in a variety of ways, as is outlined herein. In many cases multiple concentrations and/or exposure times of candidate agents can be done. Administration can be done prior to, during or after the administration of the isotope-labeled substrate.

D. Obtaining one or more targeted molecules of interest

In practicing the methods of the invention, in one aspect, targeted molecules of interest are obtained from a cell, tissue, or organism according to methods known in the art. The methods may be specific to the particular molecule of interest. Molecules of interest may be isolated from a biological sample.

A plurality of molecules of interest may be acquired from the cell, tissue, or organism. The one or more biological samples may be one or more biological fluids. Molecules of interest also may be obtained, and optionally partially purified or isolated, from the biological sample using standard biochemical methods known in the art.

The frequency of biological sampling can vary depending on different factors. Such factors include, but are not limited to, the nature of the molecules of interest, ease and safety of sampling, synthesis and breakdown/removal rates of the molecules of interest, and the half-life of a compound (chemical entity, biological factor, already-approved drug, drug candidate, drug lead, etc.).

The molecules of interest may also be purified partially, or optionally, isolated, by conventional purification methods including high pressure liquid chromatography (HPLC), fast performance liquid chromatography (FPLC), chemical extraction, thin layer chromatography, gas chromatography, gel electrophoresis, and/or other separation methods known to those skilled in the art.

In another embodiment, the molecules of interest may be hydrolyzed or otherwise degraded to form smaller molecules. Hydrolysis methods include any method known in the art, including, but not limited to, chemical hydrolysis (such as acid hydrolysis) and biochemical hydrolysis (such as nuclease degradation). Hydrolysis or degradation may be conducted either before or after purification and/or isolation of the molecules of interest. The molecules of interest may also be partially purified, or optionally, isolated, by conventional purification methods including high performance liquid chromatography (HPLC), fast performance liquid chromatography (FPLC), gas chromatography, gel electrophoresis, and/or any other methods of separating chemical and/or biochemical compounds known to those skilled in the art.

D. Analysis

Presently available technologies (static methods) measure only composition, structure, or concentrations of molecules in a cell and do so at one point in time.

1. Mass Spectrometry

Mass spectrometers convert components of a sample into rapidly moving gaseous ions and separate them on the basis of their mass-to-charge ratios. The distributions of isotopes or isotopologues of ions, or ion fragments, may thus be used to measure the isotopic enrichment in one or more molecules of interest.

Generally, mass spectrometers include an ionization means and a mass analyzer. A number of different types of mass analyzers are known in the art. These include, but are not limited to, magnetic sector analyzers, electrostatic analyzers, quadrupoles, ion traps, time of flight mass analyzers, and fourier transform analyzers. In addition, two or more mass analyzers may be coupled (MS/MS) first to separate precursor ions, then to separate and measure gas phase fragment ions.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS is used to measure mass isotopomer abundances of organic molecules, hydrogen-labeled isotope incorporation from labeled water is amplified 3 to 7-fold, depending on the number of hydrogen atoms incorporated into the organic molecule from labeled water.

In one embodiment, isotope enrichments of molecules of interest may be measured directly by mass spectrometry.

In another embodiment, the molecules of interest may be partially purified, or optionally isolated, prior to mass spectral analysis. Furthermore, hydrolysis or degradation products of molecules of interest may be purified.

In another embodiment, isotope enrichments of molecules of interest after hydrolysis of the molecule of interest are measured by gas chromatography-mass spectrometry.

In each of the above embodiments the biosynthesis rate of the biological molecule (i.e., molecule of interest) can be calculated by application of the precursor-product relationship (discussed further, infra) using either labeled precursor molecule enrichment values or asymptotic isotope enrichment of a fully turned over molecule of interest to represent the true precursor pool enrichment. Alternatively, the biosynthesis or breakdown rate may be calculated using an exponential decay curve by application of exponential or other die-away kinetic models (discussed further, infra).

a. Measuring Relative and Absolute Mass Isotopomer Abundances

Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the present invention.

2. Calculating Labeled: Unlabeled Proportion of Molecules of Interest

The proportion of labeled and unlabeled molecules of interest is then calculated. The practitioner first determines measured excess molar ratios for isolated isotopomer species of a molecule. The practitioner then compares measured internal pattern of excess ratios to the theoretical patterns. Such theoretical patterns can be calculated using the binomial or multinomial distribution relationships as described in U.S. Pat. Nos. 5,338,686, 5,910,403, and 6,010,846, which are hereby incorporated by reference in their entirety. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California, Berkeley.

The comparison of excess molar ratios to the theoretical patterns can be carried out using a table generated for a molecule of interest, or graphically, using determined relationships. From these comparisons, a value, such as the value p, is determined, which describes the probability of mass isotopic enrichment of a subunit in a precursor subunit pool. This enrichment is then used to determine a value, such as the value Aₓ*, which describes the enrichment of newly synthesized proteins for each mass isotopomer, to reveal the isotopomer excess ratio which would be expected to be present, if all isotopomers were newly synthesized.

Fractional abundances are then calculated. Fractional abundances of individual isotopes (for elements) or mass isotopomers (for molecules) are the fraction of the total abundance represented by that particular isotope or mass isotopomer. This is distinguished from relative abundance, wherein the most abundant species is given the value 100 and all other species are normalized relative to 100 and expressed as percent relative abundance. For a mass isotopomer M_{X},

Fractional abundance of

$${M}_{x} = {A}_{x} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

Δ Fractional abundance (enrichment or depletion)=

$$\begin{matrix}\begin{matrix}Δ Fractional abundance \\ ( enrichment or depletion )\end{matrix} = {( {A}_{x} )}_{e} - {( {A}_{x} )}_{b} \\ = {( \frac{Abundance {M}_{x}}{{\sum }_{i = 0}^{n} Abundance {M}_{i}} )}_{e} - {( \frac{Abundance {M}_{x}}{{\sum }_{i = 0}^{n} Abundance {M}_{i}} )}_{b} ,\end{matrix}$$

where subscript e refers to enriched and b refers to baseline or natural abundance.

In order to determine the fraction of polymers that were actually newly synthesized during a period of precursor administration, the measured excess molar ratio (EMₓ) is compared to the calculated enrichment value, Aₓ*, which describes the enrichment of newly synthesized biopolymers for each mass isotopomer, to reveal the isotopomer excess ratio which would be expected to be present, if all isotopomers were newly synthesized.

3. Calculating Molecular Flux Rates

The method of determining rate of synthesis includes calculating the proportion of mass isotopically labeled subunit present in the molecular precursor pool, and using this proportion to calculate an expected frequency of a molecule of interest containing at least one mass isotopically labeled subunit. This expected frequency is then compared to the actual, experimentally determined isotopomer frequency of the molecule of interest. From these values, the proportion of the molecule of interest which is synthesized from added isotopically labeled precursors during a selected incorporation period can be determined. Thus, the rate of synthesis during such a time period is also determined.

A precursor-product relationship may then be applied. For the continuous labeling method, the isotopic enrichment is compared to asymptotic (i.e., maximal possible) enrichment and kinetic parameters (e.g., synthesis rates) are calculated from precursor-product equations. The fractional synthesis rate (kₛ) may be determined by applying the continuous labeling, precursor-product formula:
kₛ’[−ln(1−f)]/t,
where f=fractional synthesis=product enrichment/asymptotic precursor/enrichment
and t=time of label administration of contacting in the system studied.

For the discontinuous labeling method, the rate of decline in isotope enrichment is calculated and the kinetic parameters of the molecules of interest are calculated from exponential decay equations. In practicing the method, biopolymers are enriched in mass isotopomers, preferably containing multiple mass isotopically labeled precursors. These higher mass isotopomers of the molecules of interest, e.g., molecules containing 3 or 4 mass isotopically labeled precursors, are formed in negligible amounts in the absence of exogenous precursor, due to the relatively low abundance of natural mass isotopically labeled precursor, but are formed in significant amounts during the period of molecular precursor incorporation. The molecules of interest taken from the cell, tissue, or organism at the sequential time points are analyzed by mass spectrometry, to determine the relative frequencies of a high mass isotopomer. Since the high mass isotopomer is synthesized almost exclusively before the first time point, its decay between the two time points provides a direct measure of the rate of decay of the molecule of interest.

Preferably, the first time point is at least 2-3 hours after administration of precursor has ceased, depending on mode of administration, to ensure that the proportion of mass isotopically labeled subunit has decayed substantially from its highest level following precursor administration. In one embodiment, the following time points are typically 1-4 hours after the first time point, but this timing will depend upon the replacement rate of the biopolymer pool.

The rate of decay of the molecule of interest is determined from the decay curve for the three-isotope molecule of interest. In the present case, where the decay curve is defined by several time points, the decay kinetic can be determined by fitting the curve to an exponential decay curve, and from this, determining a decay constant.

Breakdown rate constants (k_{d}) may be calculated based on an exponential or other kinetic decay curve:
k_{d}=[−ln f]/t.

As described, the method can be used to determine subunit pool composition and rates of synthesis and decay for substantially any biopolymer which is formed from two or more identical subunits which can be mass isotopically labeled. Other well-known calculation techniques and experimental labeling or de-labeling approaches can be used (e.g., see Wolfe, R. R. Radioactive and Stable Isotope Tracers in Biomedicine: Principles and Practice of Kinetic Analysis. John Wiley & Sons; (March 1992)) for calculation flux rates of molecules and flux rates through metabolic pathways of interest.

4. Measurement of RR activity with ²H₂O

Ribonucleotide reductase catalyzes the conversion of ribonucleotides to deoxyribonucleotides (FIG. 1A). A unique feature of RR is the response of nucleoside metabolism to its inhibition. When RR is inhibited, the activity of thymidine kinase is increased. This results in increased salvage of thymidine and reduced relative contribution from de novo thymidine synthesis into DNA. Traditionally, this effect is assayed in vitro by measuring the increase in the concentration of free TTP pool relative to the other deoxynucleotides. In contrast to thymidine, basal deoxyadenosine kinase (dAk) activity is low (Krygier V., J Biol Chem. 246:2752-2757 (1971)) and is not up-regulated in response to RR inhibition. The salvage of deoxyadenosine is very low in normal cells allowing the measurement of DNA synthesis from the incorporation of de novo deoxyadenosine (see supra); furthermore since dAk activity is not affected by RR inhibition it can be used as the denominator for calculating fractional de novo thymidine synthesis. The relative inhibition of RR can be inferred from the reduction in de novo thymidine incorporation relative to dA into DNA. In a preferred embodiment of the invention, once the isotopic enrichment of dA and dT have been determined (as described, supra) for both a test and control subject or sample, the ratio of dT enrichment to dA enrichment is compared. A decrease in this ratio (i.e., a decrease in dT enrichment relative to dA enrichment) indicates that RR inhibition has occurred.

E. Uses of the Methods of the Present Invention

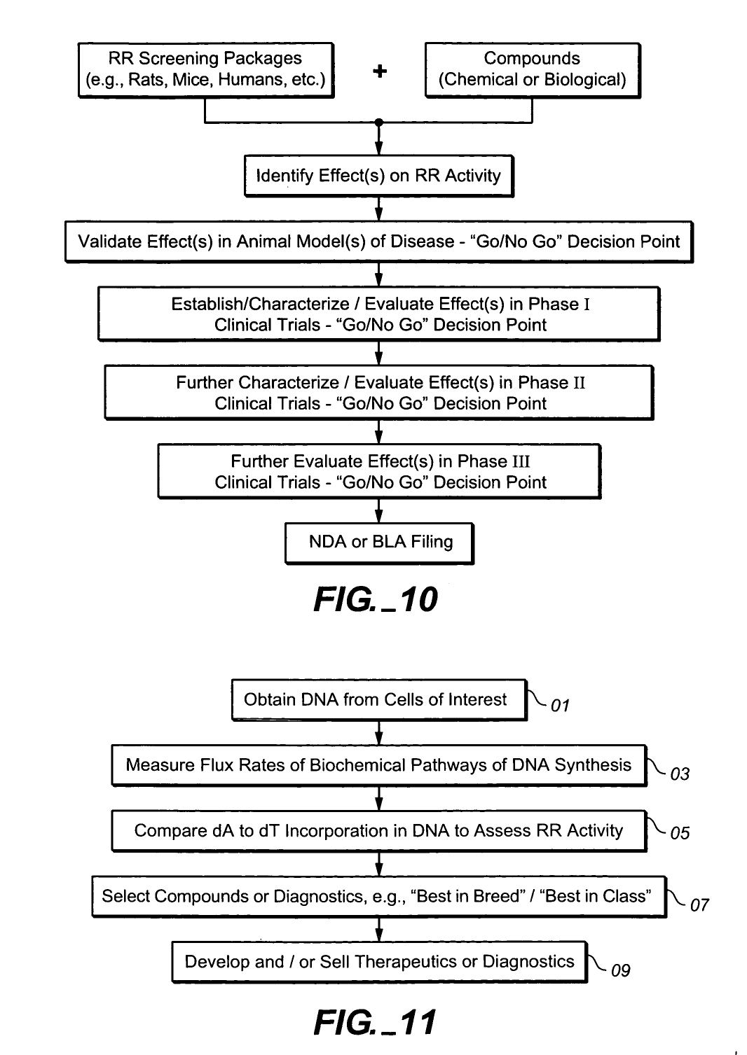

The methods disclosed herein find use in the drug discovery, development, and approval (DDDA) process (FIGS. 10-11). In particular, the methods of the present invention allow for, inter alia:
The simultaneous measurement of in vivo RR activity and cell proliferation using ²H₂O for labeling;
The use of in vivo RR activity as a pharmacodynamic biomarker of RR inhibitors;
Determining therapeutic index in vivo;
The assessing of basal thymidine salvage as it is an indicator of sensitivity to nucleoside analogs requiring kinase activation;
The quantitative comparisons of drugs, doses, and therapeutic regimes in vivo and in vitro;
A rapid, high-throughput, scaleable assay.

The methods described herein are applicable for screening candidate drug agents, FDA phase I and II human validation studies of candidate drug agents, FDA phase III approval of candidate drug agents, and FDA phase IV approval studies, or other post approval market positioning or mechanism of drug action studies.

In one embodiment, the methods allow for assessing effects on RR activity to be observed after a living system is exposed to a compound or combinations of compounds. The data generated and analyzed is therefore useful in the drug discovery, development, and approval (DDDA) process as it facilitates the DDDA decision-making process; i.e., it provides useful information for decision-makers in their decision to continue with further development on a compound or combination of compounds (e.g., if the RR inhibition data appear promising) or to cease said efforts, for example, if the RR inhibition data appear unfavorable (see FIG. 10 for a graphical depiction of this process).

Moreover, the methods allow for the skilled artisan to identify, select, and/or characterize “best in breed” in a class of compounds (i.e., “best in class”). Once identified, selected, and/or characterized, the skilled artisan, based on the information generated by the methods of the present invention, can decide to evaluate the “best in breed” further or to license the compound to another entity such as a pharmaceutical company or biotechnology company (see FIG. 11).

In another embodiment, the methods of the present invention allow for the characterization or evaluation (or both the characterization and evaluation) of toxic effects to tissues and cells from exposure to industrial chemicals, food additives, cosmetics, and environmental pollutants (e.g., an inhibition of RR activity from environmental exposure leading to a toxic injury including disease). The methods of the present invention can be used to establish programs to identify and explore the molecular mechanisms of industrial, food, cosmetic, and environmental toxicants on tissues and cells to further public health goals.

In one embodiment, data generated by the methods of the present invention may be relevant to understanding an underlying molecular pathogenesis, or causation of, one or more diseases (e.g., breast cancer or psoriasis). In another embodiment, data generated by the methods of the present invention may shed light on fundamental aspects of the initiation, progression, severity, pathology, aggressiveness, grade, activity, disability, mortality, morbidity, disease sub-classification or other underlying pathogenic or pathologic feature of a disease of interest.

In yet another embodiment, the data generated by the methods of the present invention may provide elucidation on fundamental aspects of the prognosis, survival, morbidity, mortality, stage, therapeutic response, symptomology, disability or other clinical factor of a disease of interest, particularly of diseases associated with altered cellular proliferation. Two or more biomarkers may be measured independently or concurrently (e.g., DNA synthesis and inhibition of RR activity).

Known animal models of disease may be used as part of the present invention. Such animal models of disease may include, but are not limited to, cancers of all types (including skin, breast, colon, pancreatic, brain, lung, stomach, endometrial, bladder, prostate, ovarian, uterine, leukemias, lymphomas, etc.), benign prostatic hyperplasia, inflammation, and any other proliferative disease, disorder, or condition.

In another embodiment, the methods of the invention are useful in detecting toxic effects of candidate agents such as industrial or occupational chemicals, food additives, cosmetics, or environmental pollutants/contaminants on living systems such as tissues or cells. Toxicity in the context of the present invention is usually measured by an undesirable alteration in cell proliferation, due to modulation of RR activity. As outlined herein, the modulation can either be stimulation or inhibition. In some embodiments, toxic effects may include end-organ toxicity. End-organ toxicity may include, but is not limited to, breast epithelial cell proliferation, colon epithelial cell proliferation, prostate epithelial cell proliferation, ovarian epithelial cell proliferation, bronchial epithelial cell proliferation, pancreatic epithelial cell proliferation, bladder epithelial cell proliferation, and keratinocyte proliferation.

FIG. 11 illustrates the use of the inventions herein in a drug discovery process. At step 01 a plurality of candidate agents are selected. At step 03 the flux rate of DNA synthesis is studied within cells, preferably according to the methods discussed herein. In alternative embodiments, step 03 is conducted first when the inventions are used, for example, in a target discovery process. At step 05 relevant flux rates are identified. For example, if it is desirable to reduce the flux rate of a particular biomarker of cell proliferation in a particular phenotypic state, a compound that reduces that flux rate will be considered generally more useful, and conversely a compound that increases that flux rate will be considered generally less desirable (e.g., de novo DNA synthesis). In a target discovery process, a particular phenotype that has increased or decreased flux rates with respect to another phenotype (e.g., diseased vs. not diseased) may be considered a good therapeutic or diagnostic target or in the pathway of a good therapeutic or diagnostic target. At step 07 compounds of interest, targets of interest, or diagnostics are selected and further used and further developed. In the case of targets, such targets may be the subject of, for example, well known small molecule screening processes (e.g., high-throughput screening of new chemical entities) and the like. Alternatively, biological factors, or already-approved drugs, or other compounds (or combinations and/or mixtures of compounds) may be used. At step 09 the compounds or diagnostics are sold or distributed. What is sold or distributed may be “best in breed,” so identified by the methods of the present invention. It is recognized of course that one or more of the steps in the process in FIG. 11 will be repeated many times in most cases for optimal results.

F. Isotopically-Perturbed Molecules

In another variation, the methods provide for the production of isotopically-perturbed molecules (e.g., nucleic acids). These isotopically-perturbed molecules comprise information useful in determining RR activity such as inhibition of RR activity. Once isolated from a cell and/or a tissue of an organism, one or more isotopically-perturbed molecules are analyzed to extract information as described, supra.

G. Kits

The invention also provides kits for measuring changes in RR activity. The kits may include isotope-labeled precursor molecules, and may additionally include chemical compounds known in the art for separating, purifying, or isolating proteins, and/or chemicals necessary to obtain a tissue sample, automated calculation software for combinatorial analysis, and instructions for use of the kit.

Other kit components, such as tools for administration of water (e.g., measuring cup, needles, syringes, pipettes, IV tubing), may optionally be provided in the kit. Similarly, instruments for obtaining samples from the cell, tissue, or organism (e.g., specimen cups, needles, syringes, and tissue sampling devices) may also be optionally provided.

H. Information Storage Devices

The invention also provides for information storage devices such as paper reports or data storage devices comprising data collected from the methods of the present invention. An information storage device includes, but is not limited to, written reports on paper or similar tangible medium, written reports on plastic transparency sheets or microfiche, and data stored on optical or magnetic media (e.g., compact discs, digital video discs, optical discs, magnetic discs, and the like), or computers storing the information whether temporarily or permanently. The data may be at least partially contained within a computer and may be in the form of an electronic mail message or attached to an electronic mail message as a separate electronic file. The data within the information storage devices may be “raw” (i.e., collected but unanalyzed), partially analyzed, or completely analyzed. Data analysis may be by way of computer or some other automated device or may be done manually. The information storage device may be used to download the data onto a separate data storage system (e.g., computer, hand-held computer, and the like) for further analysis or for display or both. Alternatively, the data within the information storage device may be printed onto paper, plastic transparency sheets, or other similar tangible medium for further analysis or for display or both.

### I. EXAMPLES

The following non-limiting examples further illustrate the invention disclosed herein:

### Example 1

### Measurements of Cell Proliferation in Hydroxyurea-Treated MCF7 Cells

Introduction:

As discussed, supra, the inhibition of RR is a clincally relevant mechanism of action for the treatment of many conditions, including some cancers. Many cancer drugs are evaluated in cell culture systems prior to their evaluation in animal models. Most of these cell culture models rely on tumor cell lines, which are “immortal” and fast-growing. MCF7 cells are derived from a mammary tumor, and are a commonly used cell type that can be used for in vitro (cell culture) evaluation of therapeutics, or can be implanted into mice to create tumors for in vivo evaluation of therapeutics. Hydroxyurea is a known RR inhibitor, and has strong activity in cell culture and in vivo.

Methods:

Mammary carcinoma cells (MCF7) were grown under standard conditions to approximately 50% confluence. Media was removed and replaced with media containing 10% ²H₂O, 10 □M dN's and hydroxyurea at various concentrations. Cells continued to grow for 24 hours after which they were harvested, counted by coulter counter, and frozen.

Genomic DNA was isolated from frozen cells using a Qiagen Tissue Prep Kit according to the manufacturer's instructions. DNA digestion was performed after measuring the ratio of 260/280 to quantify the amount of isolated DNA. One □g of DNA was incubated with snake venom phosphodiesterase (SVPD), deoxyribonuclease I (DNase I), nuclease P1 (NP1), and alkaline phosphatase (AP) in a digestion buffer at 37° C. for 2 h. After digestion, the reaction mixtures were filtered through a Ultrafree-MC membrane by centrifugation to remove the enzymes.

HPLC Purification: dA and T were then isolated at room temperature by using a Waters HPLC system coupled with a UV detector. The HPLC column used was a Phenomenex ODS C18 protected by an Ultrasphere ODS guard column. The mobile phase consisted of aqueous methanol at 1 ml/min flow rate. Normal nucleosides (dC, dG, dT, dA) were detected by UV absorption at 254 nm. Both thymidine and deoxyadenosine were then dried under vacuum, and stored at −20° C. Chemical Derivatization: The derivatization of the deoxyribose moiety from T and dA required different strategies, as T is resistant to hydrolysis by PFBHA whereas dA is not. 1′-phosphodeoxyribose (1′-PdR) was released from thymidine upon incubation with purified thymidine phosphorylase. 1′-PdR and dA were then derivatized directly by a pentafluorobenzylhydroxylamine (PFBHA) solution followed by acetylation with acetic anhydride.

GC-MS Analysis: Derivatization of dR (from dA) or 1′-PdR (from dT) with PFBHA yields a volatile, electron-capture derivative that is readily detected by gas chromatography coupled with NCl-MS. The analysis was performed with a DB-225 column (30-m, 0.25 mm id, 0.25 μm film thickness, J&W Scientific, Folsom, Calif.). Gas chromatography conditions were as follows: The injector temperature was held at 220° C. After 2 minutes at 100° C., the oven temperature was increased to 220° C. at a rate of 30° C./min. The temperature was then held a 220° C. for 10 min. The mass spectrometry used was negative chemical ionization, with helium as the carrier gas and methane as the reagent gas. Selective ion monitoring was used with m/z 435/436/437 of the mass isotopomers of [M-20] representing a loss of hydrofluoric acid from the molecular ion. The enrichment in M1 (EM₁) of dR was calculated by subtracting the % M1[M1/(M0+M1+M2)] of an unlabeled standard from the % M1 sample The analysis is the same for either dR or 1′-PdR, as these two molecules will yield the same derivative and masses.

Cell counts were conducted on a Coulter Counter using standard techniques well known in the art.

Results:

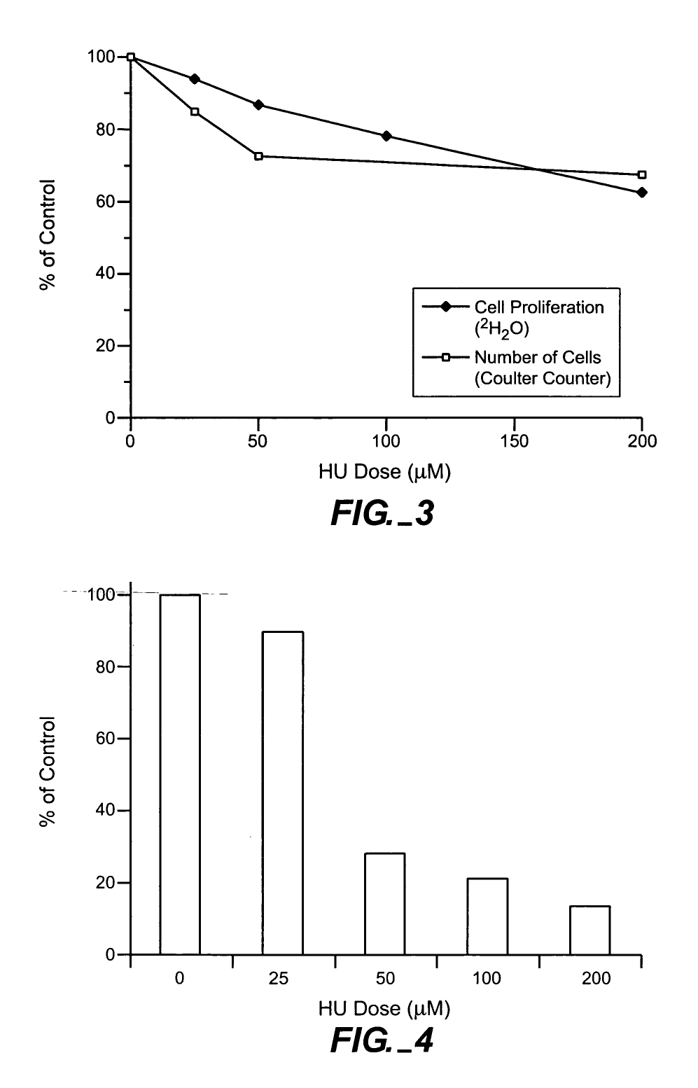

As FIGS. 3 and 4 show, hydroxyurea (HU) reduces the rate of cell proliferation, and also the activity of RR. FIG. 3 shows the comparison between a known cell-counting technique (coulter counter) and ²H₂O labeling technique. The ability of HU to inhibit cell growth is well known, and the effect is observed here using the coulter counter to count the cells. Measuring cell proliferation using ²H₂O, however, is an equally sensitive technique that shows clearer dose dependence.

The ability of HU to inhibit RR is well known, but its activity has never been measured in this manner. FIG. 4 shows the inhibition of RR by HU as measured using the present invention. Dose dependence is observed, and the ability of the technique to measure RR activity is clear. The observed effect size of HU is much larger when RR activity is measured, as opposed to cell proliferation (FIG. 3), reflecting the fact that direct RR activity measurement is superior for evaluating RR-targeted therapeutics in vitro or in vivo.

This data clearly shows that the methods and techniques of the present invention are well-suited for measuring the direct activity of an RR inhibitor (RR activity) and the indirect activity of an RR inhibitor (cell proliferation) in an in vitro cell culture system.

### Example 2

### Cell Proliferation and RR Inhibition Compared in SW1573 Cells

Introduction:

SW1573 cells are lung cancer-derived and are also frequently used for in vitro studies (see introduction in Example 1, supra). The present invention was applied to the study of these cells as well. In this case, two anti-RR therapeutics, HU and gemcitabine were used, allowing for a comparison of the two to be made.

Methods:

Non-small cell lung carcinoma cells (SW1573) were grown under standard conditions to approximately 50% confluence. Media was removed and replaced with media containing 4% ²H₂O, 1 □M dN's and hydroxyurea or gemcitabine at various concentrations. Cells were grown for and additional 24 hours after which they were harvested and frozen. Cell proliferation and RR inhibition was performed as described, supra.

Results:

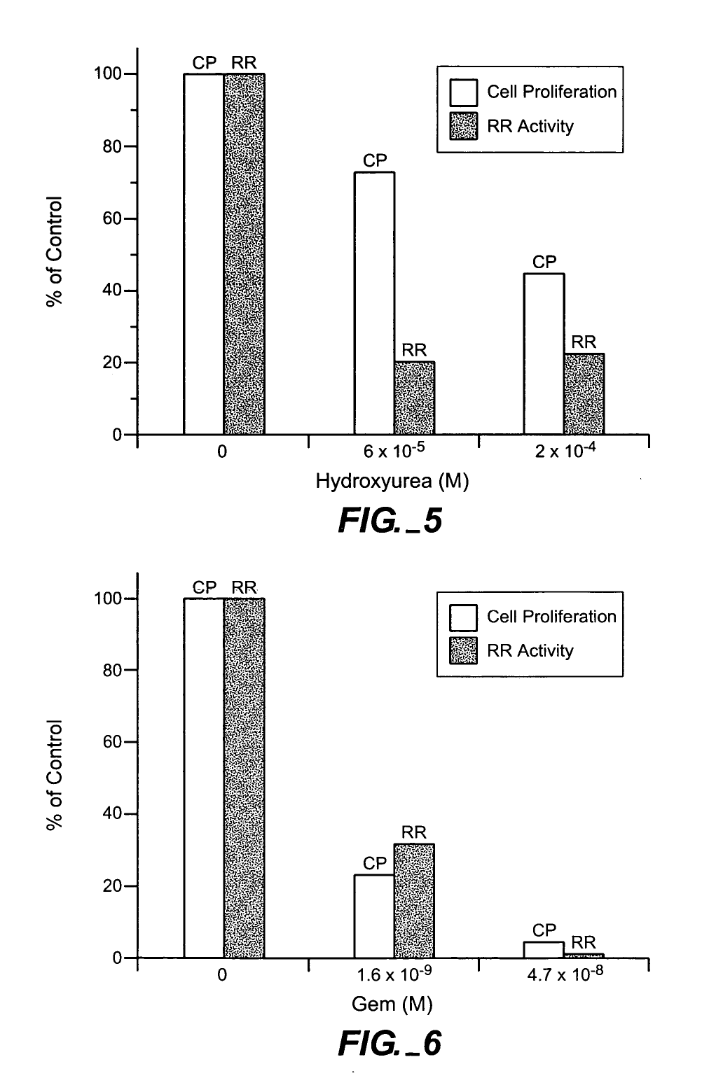

FIGS. 4 and 5 show the effect of HU and gemcitabine on the proliferation and RR activity of cultured SW1573 cells. As FIG. 5 shows, apparent maximal RR inhibition occurred at 6 □M hydroxyurea whereas inhibition of cell proliferation was evident at 20 □M. Interestingly, increasing doses of HU do not increase the inhibition of RR, which stays at a value of −20 % of control even with a nearly 30-fold increase in dose. In contrast, gemcitabine has the ability to suppress RR activity to nearly 0% of control, and clear dose dependence is observed (FIG. 6). While some dose dependence in cell proliferation is observed for HU, it does not appear to be capable of 100% suppression of RR activity, while gemcitabine is. The comparison of both RR activity data and cell proliferation data allows for the comparison of these two therapeutics. In this case, the superior ability of gemcitabine to inhibit RR and cell growth is apparent, and such data could form the basis of a decision to advance a candidate molecule for further development.

This example and Example 1 demonstrate the utility of measuring RR activity and inhibition in cell culture. Although the techniques are combined in these examples, they can also be used independently if necessary, for instance, to increase the speed at which molecules can be evaluated. The evaluation of cell proliferation need not be determined in order to determine the RR activity.

More importantly, as shown below, the technique is easily applied to in vivo models of cancer.

### Example 3

### Determination of RR Inhibition in Mouse Tumor Cells and Bone Marrow Cells Over 5 Days of Chemotherapy Treatment

Introduction:

Immortal or cancer-derived cell lines can be implanted into mice in order to grow tumors. This implantation of tumor cells creates an in vivo model of cancer that is widely used. EMT7 cells are mammary-tumor derived.

Methods:

Female balb/C mice were implanted subcutaneously with approximately 10⁶ EMT7 mouse mammary carcinoma cells in matrigel and allowed to reach ca.1500 mm³.

Mice were labeled with 2H₂O with an i.p. bolus and treatment with either 125 mg/kg gemcitabine (Gem) or 500 mg/kg HU began. Gem was administered every other day, HU daily. At then end of 5 days tumors were removed, measured, homogenized and DNA was isolated as described, supra. Bone marrow was isolated using standard techniques. Bone marrow DNA was isolated as described, supra. GC/MS analysis was described as carried out, and calculations of RR activity and cell proliferation were carried out for both tumor and bone marrow tissues. Tumor volume was also measured by use of a caliper as is standard in the art.

Results:

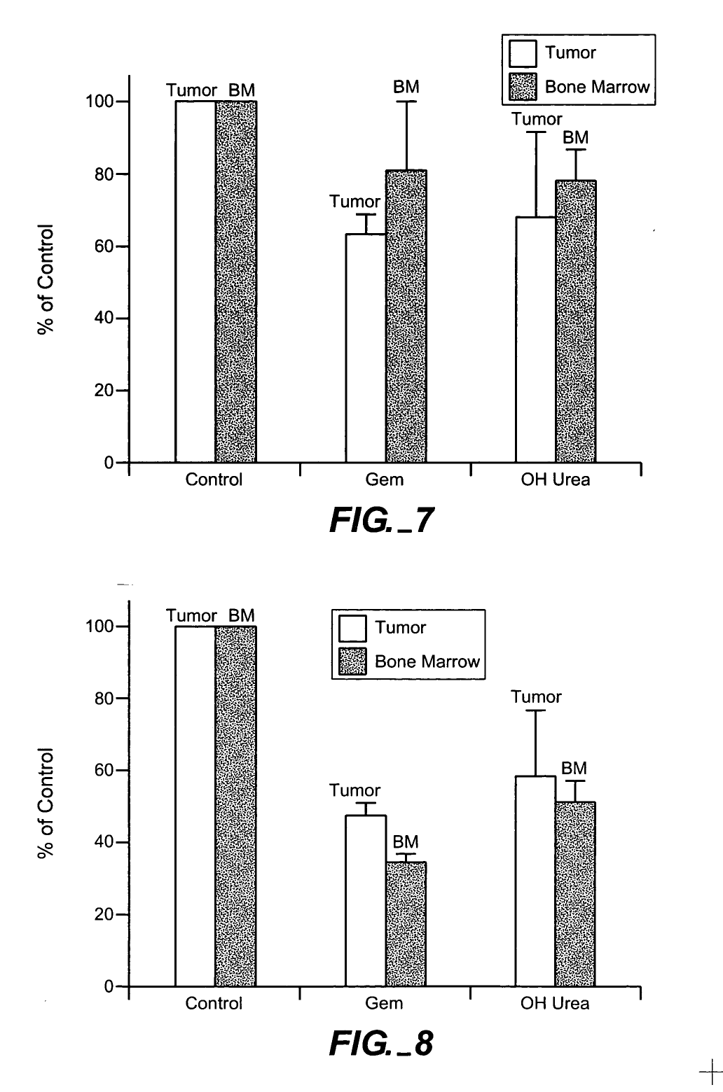

As FIG. 7 depicts, both tumor cell and bone marrow cell RR activity was inhibited after mice were treated with gemcitabine and hydroxyurea relative to untreated (control) mice. This data clearly shows that the present invention can be used to evaluate the activity of a potential therapeutic in vivo.

As FIG. 8 depicts, the effect of HU and Gem on cell proliferation is observed using the ²H₂O incorporation technique.

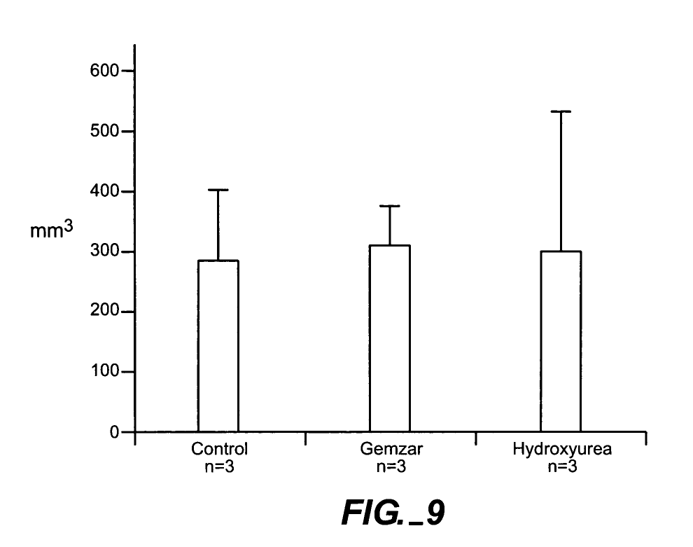

As FIG. 9 depicts, there was no significant change in tumor volume between any of the treatment groups. This is in stark contrast to the observation of reduced proliferation or RR activity, and is evidence of the improved sensitivity of the present invention. As tumor volume measurements are routinely used to evaluate potential cancer therapeutics, including RR inhibitors, this is a ground-breaking improvement.

The present invention could be used to decide if a potential therapeutic should be advanced into other stage of clinical development, or could be used to choose which candidate therapeutic was the most effective among those tested. This example shows the comparison of two different therapeutic molecules (HU and Gemcitabine) in a widely accepted in vivo model of cancer, and such evaluations can easily be carried out on large numbers of compounds to compare them all, and pick one or a few that are the most active.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes to the same extent as if each individual publication, patent, or patent application were specifically and individually indicated to be so incorporated by reference.

1. A method for measuring ribonucleotide reductase activity in a living system, said method comprising:
a) administering an isotope-labeled substrate to said living system for a first period of time sufficient for said substrate to be incorporated into at least one type of purine deoxynucleotide and at least one type of pyrimidine deoxynucleotides, to form labeled purine and pyrimidine deoxynucleotides, respectively, within a DNA molecule in said living system;
b) obtaining a first sample from said living system;
c) quantifying the amount of labeled purine deoxynucleotides and labeled pyrimidine deoxynucleotides derived from said DNA derived from said sample;
d) comparing the amount of labeled purine and pyrimidine deoxynucleotides to the amount of labeled purine and pyrimidine deoxynucleotides observed in a control living system to determine a difference in ribonucleotide reductase activity in said living system as compared to said control.

2. A method according to claim 1 further comprises calculating the molecular flux rates of said labeled purine deoxynucleotides and said labeled pyrimidine deoxynucleotides, wherein said comparing step comprises calculating the ratio of said rates and comparing said ratio to the ratio of molecular flux rates in said control living system.

3. A method according to claim 1 wherein the pyrimidine deoxynucleotide is deoxythymidine.

4. A method according to claim 1 wherein purine deoxynucleotide is deoxyadenosine.

5. A method according to claim 1 further comprising administering a candidate agent to said living system.

6. A method according to claim 5 wherein said candidate agent is administered prior to said administration of said isotope-labeled substrate.

7. A method according to claim 5 wherein said candidate agent is administered during said administration of said isotope-labeled substrate.

8. A method according to claim 5 wherein said candidate agent is administered after said administration of said isotope-labeled substrate.

9. A method according to claim 1 further comprising administering said substrate for a second period of time and repeating steps b)-d).

10. A method according to claim 1 further comprising obtaining a second sample and repeating steps c)-d).

11. A methods according to claim 5 wherein said candidate agent is a drug.

12. A method according to claim 1 wherein said ratio of said control living system is determined using steps a) to d).

13. The method of claim 1, wherein said isotope-labeled substrate is labeled with a stable isotope.

14. The method of claim 1, wherein said isotope-labeled substrate is stable-isotope labeled water.

15. The method of claim 14, wherein said stable-isotope labeled water is labeled with ²H.

16. The method of claim 1, wherein said isotope-labeled substrate is labeled with a radioactive isotope.

17. The method of claim 1, wherein said isotope-labeled substrate is radioactive-isotope labeled water.

18. The method of claim 17, wherein said radioactive isotope-labeled water is labeled with ³H.

19. The method of claim 1, wherein said living system and said control living system are mammals.

20. The method of claim 1, wherein said living system and said control living system are human.

21. The method of claim 1, wherein said living system and said control living system are cell lines.

22. The method of claim 1, wherein said living system and said control living system are primary cells.
